(19) United States
(12) Patent Application Publication
(10) Pub. No.: US 2015/0374326 A1
(43) Pub. Date: Dec. 31, 2015

(54) X-RAY DIAGNOSTIC APPARATUS

(71) Applicants: KABUSHIKI KAISHA TOSHIBA, Minato-ku (JP); Toshiba Medical Systems Corporation, Otawara-shi (JP)

(72) Inventors: Hayato KASAOKA, Nasushiobara (JP); Shunichiro NISHIGAKI, Otawara (JP); Takeo MATSUZAKI, Nasushiobara (JP); Kayoko NIHEI, Nasushiobara (JP)

(73) Assignees: KABUSHIKI KAISHA TOSHIBA, Minato-ku (JP); Toshiba Medical Systems Corporation, Otawara-shi (JP)

(21) Appl. No.: 14/843,027

(22) Filed: Sep. 2, 2015

Related U.S. Application Data
(63) Continuation of application No. PCT/JP2014/055236, filed on Mar. 3, 2014.

(30) Foreign Application Priority Data
Mar. 8, 2013 (JP) 2013-046530
Feb. 27, 2014 (JP) 2014-037268

(51) Int. Cl. A61B 6/00; A61B 6/04

(52) U.S. Cl. CPC A61B 6/547; A61B 6/4441; A61B 6/0457

(57) ABSTRACT

According to embodiment, an X-ray diagnostic apparatus includes a frame holding an X-ray tube and detector such that an axis connecting a X-ray focus and center of the detector rotates around an isocenter, display displaying a first image, operation circuitry inputting a position of a target region on the image, target position calculation circuitry calculating a position of the region on a second image based on an angle between the axes concerning the first and second images, a first distance from the isocenter to a top plate, and second distance from the target position to the plate, and top plate movement amount calculation circuitry calculating a top plate movement amount to display the region at a same position as the target position on the second image based on the calculated position, target position, angle, and first distance.

### CROSS-REFERENCE TO RELATED APPLICATIONS

This application is a Continuation application of PCT Application No. PCT/JP2014/055236, filed Mar. 3, 2014 and based upon and claims the benefit of priority from the Japanese Patent Application No. 2013-046530, filed Mar. 8, 2013, and the Japanese Patent Application No. 2014-037268, filed Feb. 27, 2014, the entire contents of all of which are incorporated herein by reference.

### FIELD

Embodiments described herein relate generally to an x-ray diagnostic apparatus.

### BACKGROUND

For example, when executing a close examination or the like by using an X-ray diagnostic apparatus having an X-ray tube and an X-ray detector arranged to face each other, like an X-ray diagnostic apparatus including a C-arm, the operator finely adjusts the angle of the C-arm while seeing an X-ray fluoroscopic image on a monitor to observe the state of a target lesion of an object from many directions.

More specifically, in order to observe a lesion from many directions, it is necessary to change the posture (angle) of the C-arm by rotating/driving it. When the posture of the C-arm is changed by rotating/driving the C-arm in this manner, the position of a target lesion on the X-ray fluoroscopic image displayed on the monitor sometimes shifts.

When, for example, a target lesion is displayed near the center of the monitor before the C-arm is rotated/driven, the target lesion is sometimes displayed at a position shifted from near the center of the monitor after the C-arm is rotated/driven.

In order to locate the target lesion at a desired position (e.g., near the center of the monitor) again after the display position on the monitor has shifted in this manner, it is necessary to move the top plate and the imaging system, and the processing of calculating the movement amounts of them is complicated. This increases the loss of time and exposure dose which are required to locate the target lesion at the desired position again.

In consideration of such situations, there is proposed a technique for preventing the shift of an imaging position accompanying a change in the angle of an X-ray axis connecting the X-ray tube to the X-ray detector.

More specifically, there is disclosed an X-ray diagnostic apparatus comprising an imaging system support means having an X-ray tube for X-ray irradiation and an X-ray detector for transmitted X-ray detection arranged to face each other through a top plate on which an object is placed, an X-ray imaging system driving means for changing the angle or position of an X-ray axis connecting the center of the X-ray tube to the center of the X-ray detector by moving the imaging system support means such that its position is determined by a positional coordinate system with the mechanical central point (isocenter) of the apparatus being a reference point, an X-ray axis obtaining means for obtaining the position of an X-ray axis at the time of obtaining the X-ray image displayed on the screen of an image monitor, and an inter-axis intersection point obtaining means for obtaining the intersection point between two X-ray axes obtained by the X-ray axis obtaining means. This X-ray diagnostic apparatus is configured to move the imaging system support means to make the intersection point obtained by the inter-axis intersection point obtaining means always pass through the X-ray axis, when the X-ray imaging system driving means changes the angle of the X-ray axis.

A related art can only be applied to an X-ray diagnostic apparatus including a driving mechanism which can “move the imaging system support means to make the intersection point obtained by the inter-axis intersection point obtaining means always pass through the X-ray axis, when the X-ray imaging system driving means changes the angle of the X-ray axis”. An X-ray diagnostic apparatus which does not include such a driving mechanism has been put into practice.

Demands have therefore arisen for a technique of performing control to correct the shift of an imaging position by using a driving mechanism of a general commercially available X-ray diagnostic apparatus, even when a member which supports an imaging system is rotated/driven.

### BRIEF DESCRIPTION OF THE DRAWINGS

FIG. 1 is a view showing an example of the arrangement of an X-ray diagnostic apparatus according to the first embodiment.

FIG. 2A is a view showing the first portion of a flowchart for bed position correction processing by the X-ray diagnostic apparatus according to the first embodiment.

FIG. 2B is a view showing the second portion of the flowchart for bed position correction processing by the X-ray diagnostic apparatus according to the first embodiment.

FIG. 3A is a schematic view showing the positional relationship between an imaging system and an object when processing in step S1 is executed according to the first embodiment.

FIG. 3B is a view showing a display example on an image monitor when the processing in step S1 is executed according to the first embodiment.

FIG. 4A is a schematic view showing the positional relationship between the imaging system and the object when processing in step S2 is executed according to the first embodiment.

FIG. 4B is a view showing a display example on the image monitor after the processing in step S2 is executed according to the first embodiment.

FIG. 5A is a schematic view showing the positional relationship between the imaging system and the object after processing in step S3 is executed according to the first embodiment.

FIG. 5B is a view showing a display example on the image monitor after the processing in step S3 is executed according to the first embodiment.

FIG. 6A is a schematic view showing the positional relationship between the imaging system and the object after processing in step S7 is executed according to the first embodiment.

FIG. 6B is a view showing a display example on the image monitor after the processing in step S7 is executed according to the first embodiment.

FIG. 7A is a schematic view showing the positional relationship between the imaging system and the object after processing in step S10 is executed according to the first embodiment.

FIG. 7B is a view showing a display example on the image monitor after the processing in step S10 is executed according to the first embodiment.

FIG. 8 is a view concerning the calculation of top plate movement amounts according to the second embodiment.

FIG. 9 is a view for explaining the calculation of top plate movement amounts according to the second embodiment.

FIG. 10 is a view for explaining the calculation of top plate movement amounts according to the second embodiment.

FIG. 11 is a flowchart showing an example of a procedure for the operation associated with a top plate moving function according to the second embodiment.

FIG. 12 is a view for explaining the calculation of top plate movement amounts according to a modification of the second embodiment.

FIG. 13 is a flowchart showing an example of a procedure for the operation associated with the top plate moving function according to a modification of the second embodiment.

### DETAILED DESCRIPTION

In general, according to one embodiment, an X-ray diagnostic apparatus includes an X-ray tube, an X-ray detector, a holding frame, a display, operation circuitry, target position calculation circuitry, and top plate movement amount calculation circuitry.

The X-ray tube and the X-ray detector face each other.

The holding frame holds the X-ray tube and the X-ray detector such that an imaging system axis passing through a focus of the X-ray tube and a central position of the X-ray detector is configured to rotate around an isocenter as a rotation center around a top plate.

The display displays a first X-ray image obtained by the X-ray detector.

The operation circuitry inputs a target position corresponding to a target region with respect to the first X-ray image displayed on the display.

The target position calculation circuitry calculates a position corresponding to the target region with respect to a second X-ray image different from the first X-ray image based on an angle between the imaging system axis concerning the second X-ray image and the imaging system axis concerning the first X-ray image, a distance from the isocenter to the top plate, and a distance between the target position and the top plate.

The top plate movement amount calculation circuitry calculates a movement amount of the top plate to display the target region at a same position as the target position on the second X-ray image based on the calculated position, the target position, the angle, and the distance from the isocenter to the top plate.

An X-ray diagnostic apparatus according to this embodiment will be described below with reference to the accompanying drawings. Note that the same reference numerals denote constituent elements having almost the same arrangements in the following description, and a repetitive description will be made only when required.

### First Embodiment

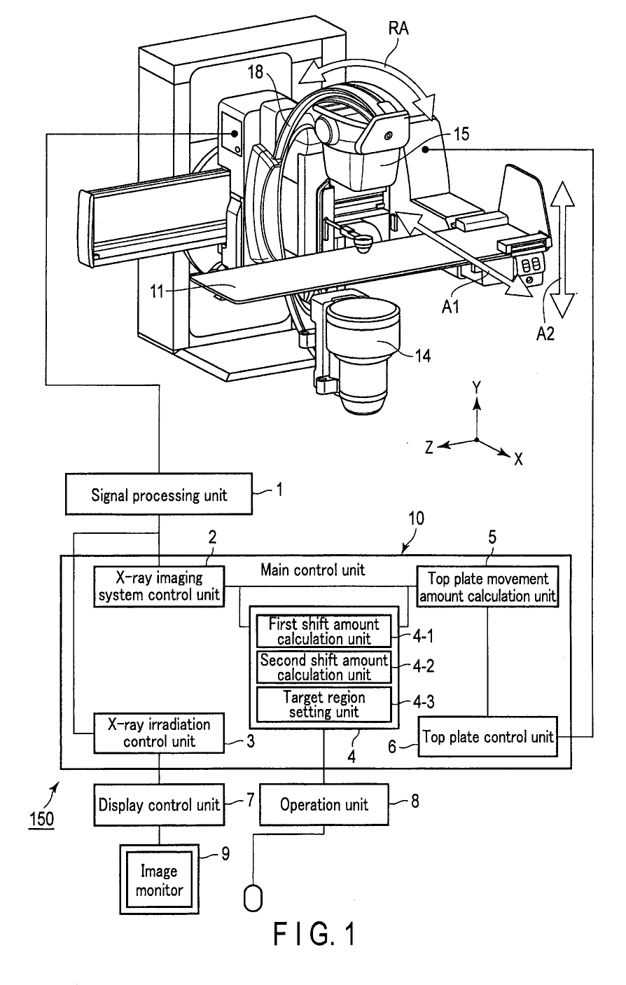

FIG. 1 is a block diagram showing an example of the arrangement of an X-ray diagnostic apparatus 150 according to the first embodiment. In the first embodiment, as shown in FIG. 1, the X-axis, the Y-axis, and the Z-axis are set. That is, the Z-axis is set in a direction parallel to the long axis of a top plate 11, the X-axis is set in a direction parallel to the short axis of the top plate 11, and the Y-axis is set in a direction perpendicular to the upper surface of the top plate 11.

The X-ray diagnostic apparatus 150 according to the first embodiment includes a signal processing unit 1, a display control unit 7, an operation unit 8, an image monitor 9, a main control unit 10, the top plate 11, an X-ray irradiation unit 14, an X-ray detector 15, a C-arm (holding unit) 18, and an X-ray imaging unit. The X-ray imaging unit includes an X-ray tube and an X-ray detector which face each other.

The signal processing unit 1 generates X-ray image data by processing the X-ray detection signal generated by the X-ray detector 15. In addition, the signal processing unit 1 generates each piece of axis position information by processing an encoder signal originating from each bed operation axis.

The main control unit 10 includes an X-ray imaging system control unit 2, an x-ray irradiation control unit 3, a target position calculation unit 4, a top plate movement amount calculation unit 5, and a top plate control unit 6.

The X-ray imaging system control unit 2 performs control to obtain an X-ray image of an object by rotating the C-arm 18, around the isocenter, which supports the X-ray irradiation unit 14 which irradiates the object with X-rays and the X-ray detector 15 which detects the X-rays transmitted through the object. The isocenter is the rotation center of the C-arm 18.

The X-ray irradiation control unit 3 controls the X-ray irradiation unit 14 so as to irradiate an object on the top plate 11 with predetermined X-rays.

The target position calculation unit 4 includes a first shift amount calculation unit 4-1 which calculates a first shift amount L1, a second shift amount calculation unit 4-2 which calculates a second shift amount L2, and a target region setting unit 4-3 which sets the region designated by using the operation unit 8 as a target region.

The first shift amount L1 is the distance between a “re-designated” target region (to be describe later) of an object and a target position (a desired position at which the target region is to be located) on the X-axis (along the X-axis direction). The second shift amount L2 is the distance (i.e., depth information) between a “re-designated” target region of an object and the isocenter described above on the Y-axis (along the Y-axis direction or an imaging system axis (to be described later)). A concrete method of calculating the first shift amount L1 and the second shift amount L2 will be described in detail later.

Note that the above “target position” is set in advance by the user and recorded on a memory (not shown) of the main control unit 10. In this case, the central position of the X-ray image displayed on the image monitor 9 is set as a target position. The first shift amount L1 is therefore the distance between the “re-designated” target region of an object and the imaging system axis (the central position of the X-ray image displayed on the image monitor 9) (to be described later) on the X-axis.

The top plate movement amount calculation unit 5 calculates the movement amounts of the top plate 11 which are required to locate the target region of the object at the target position on the X-ray image displayed on the image monitor 9, based on the first shift amount and the second shift amount. A concrete method of calculating movement amounts by the top plate movement amount calculation unit 5 will be described in detail later.

The top plate control unit 6 controls the vertical movement of the top plate 11 in the up-down direction (Y direction) and the horizontal movement of the top plate 11 in the transverse direction (X direction). That is, the top plate control unit 6 performs driving control of the top plate 11.

The display control unit 7 causes the image monitor 9 to display the X-ray image generated by the signal processing unit 1. In other words, the image monitor 9 displays X-ray images under the control of the display control unit 7.

The operation unit 8 is, for example, a control panel, footswitch, or joystick, and accepts the input of various types of operations with respect to the X-ray diagnostic apparatus 150 from the operator. More specifically, the operation unit 8 accepts, for example, an instruction to acquire X-ray image data and various types of operation instructions. For example, the operation unit 8 accepts the operation of moving the top plate 11, the operation of rotating the C-arm 18, and the operation of executing X-ray imaging. The top plate control unit 6, X-ray imaging system control unit 2, and X-ray irradiation control unit 3 of the main control unit 10 perform control concerning operations corresponding to the various types of operations accepted by the operation unit 8.

The operation unit 8 functions as a designation unit for designating a target position which is a position at which a target region of an object is to be displayed on the X-ray image displayed on the image monitor 9.

The operation unit 8 functions as a re-designation unit for designating a target region of an object (setting “re-designated position” to be described later) on the X-ray image displayed on the image monitor 9 after the rotating/driving of the C-arm 18.

The top plate 11 is configured to be capable of performing a top plate horizontal operation (movement along the X-axis) in the direction indicated by a double-headed arrow A1 in FIG. 1 and a top plate vertical operation (movement along the Y-axis) in the direction indicated by a double-headed arrow A2 in FIG. 1. An object is placed on the top plate 11.

The X-ray irradiation unit 14 includes an X-ray tube which emits X-rays. The X-ray detector 15 detects the X-rays emitted from the X-ray tube and transmitted through an object. The pair of the X-ray irradiation unit 14 and the X-ray detector 15 is configured to rotate around a geometrical rotation center. This rotation center is the isocenter. In this case, the axis obtained by connecting the center of the X-ray tube (the X-ray focus from which X-rays are generated) of the X-ray irradiation unit 14 and the center of the X-ray detector 15 (the central (barycentric) position on the detection surface of the X-ray detector 15) with a straight line is called the imaging system axis. The IC (Isocenter) as the rotation center of the C-arm 18 is located on the imaging system axis.

The C-arm (holding unit) 18 is a support unit which supports the X-ray irradiation unit 14 and the X-ray detector 15 which face each other. The C-arm 18 is configured to be capable of slidably rotating in the direction indicated by an arrow RA along the curve of the arm while rotating about the isocenter in the arm longitudinal direction.

Note that the holding unit 18 is not limited to a C-arm, and may be an Ω-arm, U-arm, or the like. In addition, the holding unit 18 may hold the X-ray tube and the X-ray detector 15 as discrete components. In this case, the holding unit 18 holds the X-ray tube and the X-ray detector 15 so as to make them face each other.

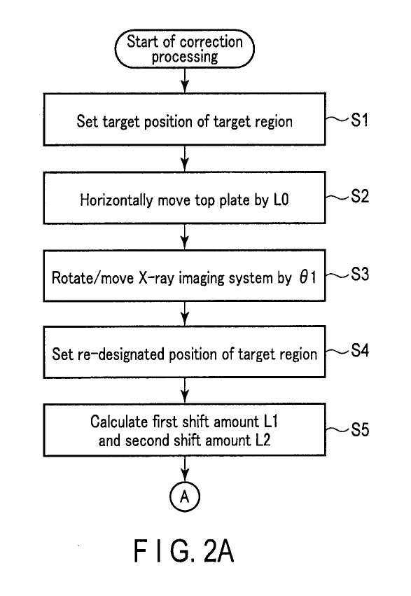

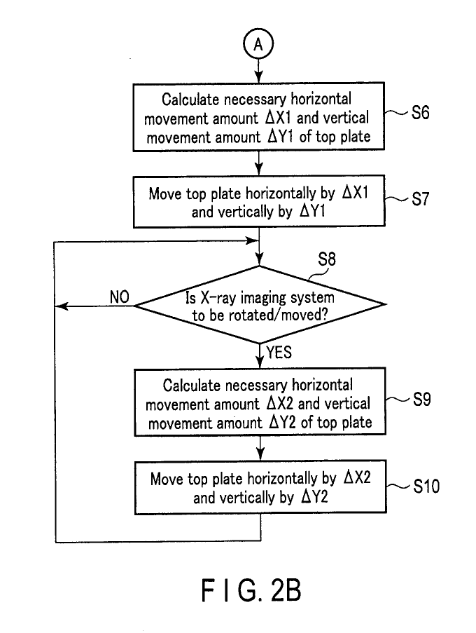

FIG. 2A and FIG. 2B is a flowchart for bed position correction processing by the X-ray diagnostic apparatus 150 according to the first embodiment. FIG. 2A shows the first portion of the flowchart for bed position correction processing by the X-ray diagnostic apparatus 150 according to the first embodiment. FIG. 2B shows the second portion of the flowchart for bed position correction processing by the X-ray diagnostic apparatus 150 according to the first embodiment.

First of all, the user designates the position of a target region 100 of an object P on the X-ray image (the obtained image, LIH (Last Image Hold) image, or the like) displayed on the image monitor 9 by using the operation unit 8. The target region setting unit 4-3 sets this designated position as the position of the target region 100 (step S1).

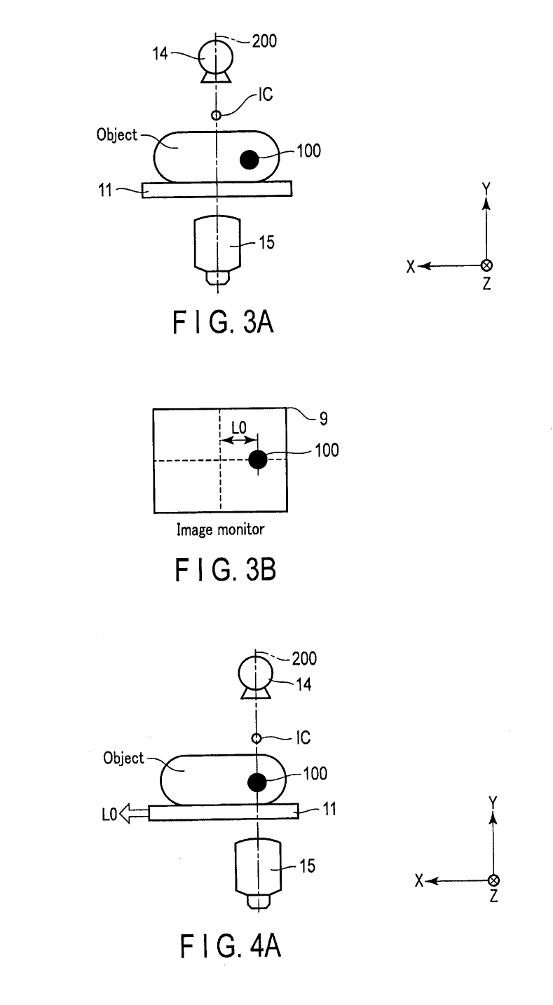

FIG. 3A is a schematic view showing the positional relationship between the imaging system and the object P at the time of the execution of processing in step S1. FIG. 3B is a view showing a display example on the image monitor 9 at the time of the execution of the processing in step S1.

In this case, as shown FIGS. 3A and 3B, the target region 100 is located at a position shifted from an imaging system axis 200 described above by a distance L0.

Subsequently, the top plate control unit 6 horizontally moves the top plate 11 in the X direction so as to locate the target region 100 set in step S1 at the position desired by the user (the central position on the image monitor 9 in this case) (step S2). FIG. 4A is a schematic view showing the positional relationship between the imaging system and the object P at the time of the execution of processing in step S2. FIG. 4B is a view showing a display example on the image monitor 9 after the execution of the processing in step S2.

In this case, the top plate control unit 6 horizontally moves the top plate 11 by the distance L0 as shown in FIG. 4A to locate the target region 100 at the target position (the central position on the image monitor 9) (on the imaging system axis 200), as shown in FIG. 4B.

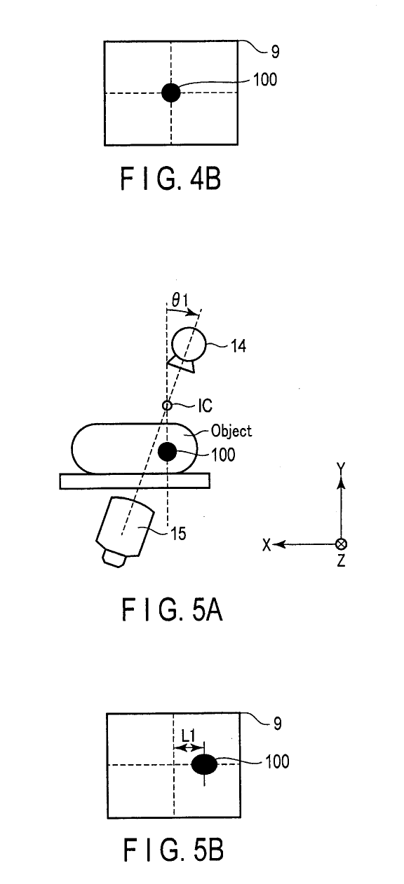

Subsequently, the user performs the operation of rotating/moving the C-arm 18 (assume that this is the operation of rotating/moving the C-arm 18 by an angle θ₁) using the operation unit 8. The X-ray imaging system control unit 2 rotates/moves the C-arm 18 by the angle θ1 in accordance with this operation (step S3). FIG. 5A is a schematic view showing the positional relationship between the imaging system and the object P after the execution of processing in step S3. FIG. 5B is a view showing an display example on the image monitor 9 after the execution of the processing in step S3.

After the completion of the processing in step S3, the target region 100 is shifted from the imaging system axis 200, as shown in FIG. 5A. That is, as shown in FIG. 5B, the target region 100 is displayed at a position shifted from the center of the image monitor 9 (the imaging system axis 200) by a distance L1.

In this case, the user designates again (to be abbreviated to “re-designates”) the position of the target region 100 of the object P on the X-ray image (the obtained image, or LIH image, or the like) displayed on the image monitor 9 by using the operation unit 8. The target region setting unit 4-3 sets this re-designated position as the re-designated position” of the target region 100 (step S4).

The first shift amount calculation unit 4-1 of the target position calculation unit 4 calculates the first shift amount L1 based on the target position and the re-designated position of the target region 100 (for example, from the distance between the target position and the re-designated position on the X-ray image). The first shift amount L1 is the distance between the re-designated target region 100 and the imaging system axis 200.

In addition, the second shift amount calculation unit 4-2 of the target position calculation unit 4 calculates the second shift amount L2 based on the first shift amount L1 and the rotation angle θ₁ (the tangent to the rotation angle) (step S5). The second shift amount L2 is the distance between the re-designated target region 100 and the isocenter IC on the Y-axis (or the imaging system axis).

More specifically, the second shift amount calculation unit 4-2 calculates the second shift amount L2 by:

L2=L1/tan θ₁

The top plate movement amount calculation unit 5 then calculates, based on the second shift amount L2 calculated in step S5, the movement amounts of the top plate 11 which are required to locate the target region 100 at the target position (the central position on the image monitor 9) on the X-ray image displayed on the image monitor 9 (step S6).

More specifically, the top plate movement amount calculation unit 5 calculates a movement amount ΔX1 of the top plate 11 in the X direction and a movement amount ΔY1 of the top plate 11 in the Y direction by:

ΔX1=L2×sin θ₁

ΔY1=L2×(1−cos θ₁)

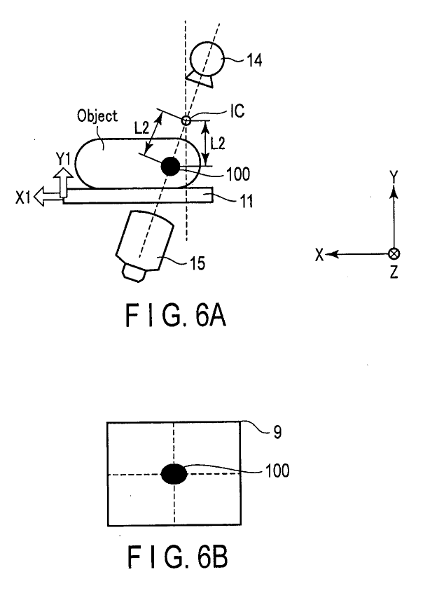

In this case, the top plate control unit 6 moves the top plate 11 by the movement amount ΔX1 in the X direction (horizontal direction) based on the calculation result obtained in step S6, and moves the top plate 11 by the movement amount ΔY1 in the Y direction (vertical direction) (step S7). FIG. 6A is a schematic view showing the positional relationship between the imaging system and the object P after the execution of processing in step S7. FIG. 6B is a view showing a display example on the image monitor 9 after the execution of the processing in step S7. As shown in FIGS. 6A and 6B, upon completion of the processing in step S7, the target region 100 is displayed at the target position (the central position on the image monitor 9) on the X-ray image displayed on the image monitor 9.

In this case, the X-ray imaging system control unit 2 determines whether a rotating/moving operation for the C-arm 18 has been performed by using the operation unit 8 (step S8). If YES in step S8 (it is determined that a rotating/moving operation for the C-arm 18 has been performed), the X-ray imaging system control unit 2 rotates/moves the C-arm 18 by an angle θ₂ (assume that a rotating/moving operation corresponding to the angle θ₂ has been performed in this case), the target position calculation unit 4 calculates the first shift amount L1 and the second shift amount L2, and the top plate movement amount calculation unit 5 calculates movement amounts ΔX2 and ΔY2 of the top plate 11 (step S9).

Note that the processing performed by the target position calculation unit 4 in step S9 and the processing performed by the top plate movement amount calculation unit 5 are similar to those in steps S5 and S6. That is, L2, ΔX2, and ΔY2 are calculated by:

L2=L1/tan θ₂

ΔX2=L2×sin θ₂

ΔY2=L2×(1−cos θ₂)

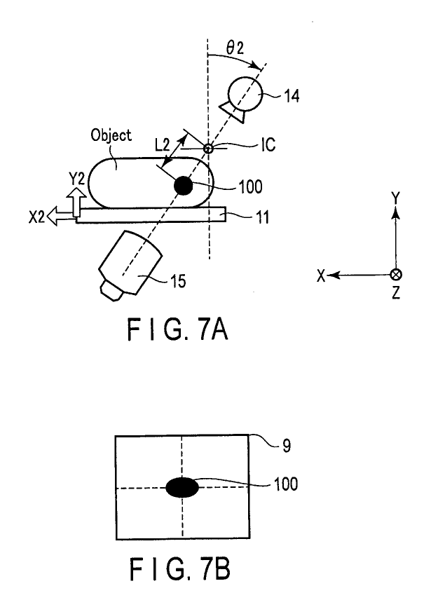

The top plate control unit 6 then moves the top plate 11 by the movement amount ΔX2 in the X direction (horizontal direction) and also moves the top plate 11 by the movement amount ΔY2 in the Y direction (vertical direction) based on the calculation result obtained in step S9 described above (step S10). FIG. 7A is a schematic view showing the positional relationship between the imaging system and the object P after the execution of processing in step S10. FIG. 7B is a view showing a display example on the image monitor 9 after the execution of the processing in step S10. As shown in FIGS. 7A and 7B, when the processing in step S10 is complete, the target region 100 is displayed at the target position (the central position on the image monitor 9) on the X-ray image displayed on the image monitor 9.

If NO in step S8 (it is determined that a rotating/moving operation for the C-arm 18 has not been performed), the process returns to step S8. That is, step S8 is the step of waiting until the execution of a rotating/moving operation for the C-arm 18.

As described above, this embodiment can provide the X-ray diagnostic apparatus 150 which corrects the shift of an imaging position at the time of rotating/driving of the member supporting the imaging system, without including any special driving mechanism. More specifically, the X-ray diagnostic apparatus 150 according to the embodiment has the following effects.

That is, when the user only performs designating and re-designating operations with respect to the target region 100 on the image monitor 9, the top plate 11 is driven/controlled to automatically display the target region 100 at a desired position (e.g., the central position on the image monitor 9). This makes it possible for the user to always display the target region 100 at the central position on the image monitor 9 by only performing an angle adjusting operation for the C-arm 18 without performing any special operation. The user can therefore concentrate on only the observation of a lesion without paying any attention to the movement of the top plate 11. In addition, shortening of the operation time can achieve reductions in exposure dose and observation field of view.

Note that the target region 100 may be displayed at a desired position (the central position on the image monitor 9) on the image monitor 9 by performing image processing for the X-ray image data based on the first shift amount L1 and the second shift amount L2 instead of moving the top plate 11.

Note that the form (mode) of the X-ray diagnostic apparatus 150 shown in FIG. 1 is merely an example, and the above embodiment can also be applied to X-ray diagnostic apparatuses of other forms (modes).

### Second Embodiment

A difference from the first embodiment is that a re-designating operation is omitted by setting the distance from the IC to the top plate as the second shift amount as a known value.

An operation unit 8 inputs a target position corresponding to a target region with respect to the first X-ray image displayed on an image monitor (display unit) 9. Note that the operation unit 8 may include a switch for turning on or off a top plate moving function (to be described later) in accordance with the operation of the user. In addition, the top plate moving function may be turned on or off based on the examination information (e.g., an examination name) output from an RIS (Radiology Information System) or HIS (Hospital Information System) via a network and an interface (neither of which is shown). The operation unit 8 inputs a rotation angle θ through which a holding unit 18 is rotated, in accordance with a support from the user.

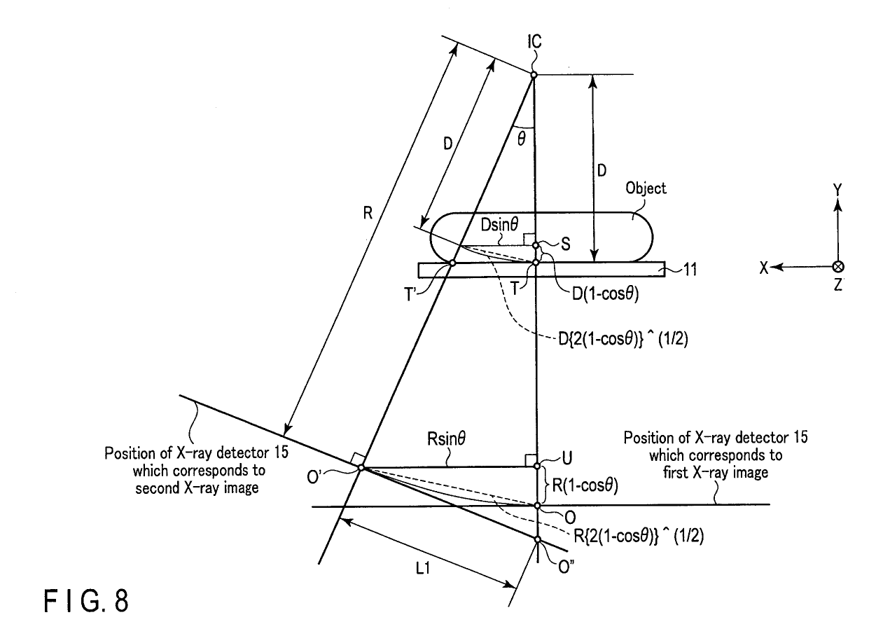

As shown in FIG. 8, since the distance from the isocenter IC to a top plate 11 is grasped as the position of the top plate vertical operation axis, which is represented by D, the movement amounts of the top plate 11 are respectively represented by D (1−cos θ) along the Y-axis direction and D sin θ along the X-axis direction.

Note that when a target position is an arbitrary position which is not the central position of an X-ray detector 15, a top plate movement amount calculation unit 5 calculates the movement amounts of the top plate 11 by correcting the movement amounts of the top plate 11 using the difference between the central position and the target position.

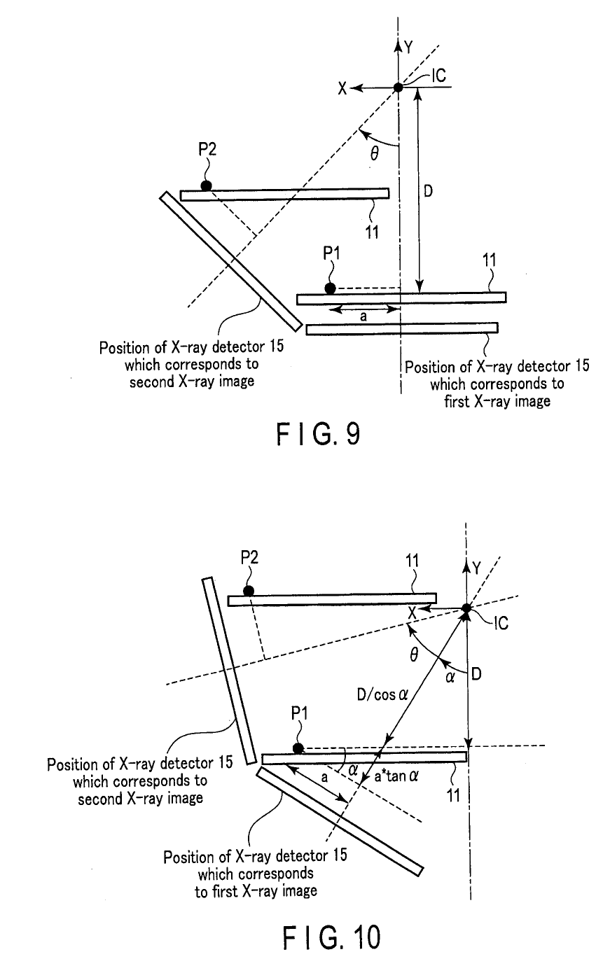

More specifically, assume that, as shown in FIG. 9, a target position P1 input with respect to the first X-ray image is a non-central position of the X-ray detector 15, the Y-axis including the isocenter IC coincides with the imaging system axis at the time of obtaining the first X-ray image, and the target position P1 is not moved to the central position by moving the top plate 11 before the rotation of the holding unit 18. In this case, the movement amounts of the top plate 11 are calculated by:

$$( \begin{matrix}X \\ Y\end{matrix} ) = ( \begin{matrix}cos θ & - sin θ \\ sin θ & cos θ\end{matrix} ) ( \begin{matrix}a \\ - D\end{matrix} ) = ( \begin{matrix}a \times cos θ + D \times sin θ \\ a \times sin θ - D \times cos θ\end{matrix} )$$

where (X, Y) represents the coordinates of a target position P2 after the rotation of the holding unit 18, (a, −D) represents the coordinates of the target position P1 at a non-central position, θ represents the rotation angle of the holding unit 18 having the isocenter IC as a rotation center, “a” represents the distance between the central position and the target position, and D represents the distance between the isocenter IC and the top plate 11, which is the length of the top plate vertical operation axis. Note that the coordinates are based on the isocenter IC as an origin.

The following equations are used to calculate the movement amounts of the top plate 11 which cause the target position P1 before the rotation of the holding unit 18 and the target position P2 after the rotation of the holding unit 18 through the rotation angle θ to be displayed at almost the same position on the monitor. A movement amount ΔX of the top plate 11 along the X direction is calculated by:

$$\begin{matrix}Δ X = X - a \\ = a \times cos θ + D \times sin θ - a \\ = a \times ( cos θ - 1 ) + D \times sin θ\end{matrix}$$

In addition, a movement amount ΔY of the top plate 11 along the Y direction is calculated by:

$$\begin{matrix}Δ Y = Y - ( - D ) \\ = a \times sin θ - D \times cos θ + D \\ = a \times sin θ - D \times ( cos θ - 1 )\end{matrix}$$

In more general, assume that, as shown in FIG. 10, the target position P1 input with respect to the first X-ray image is a non-central position of the X-ray detector 15, the Y-axis including the isocenter IC at the time of obtaining the first X-ray image differs from the imaging system axis, and the target position P1 is not moved to the central position by the movement of the top plate 11 before the rotation of the holding unit 18. In this case, the movement amounts of the top plate 11 are calculated by the following equations. A movement amount ΔX′ of the top plate 11 along the X direction at the time of the rotation of the coordinate system by α° is calculated by replacing −D with −(D/cos α+a×tan α) in the above equation for ΔX as follows:

ΔX′=a×(cos θ−1)+(D/cos α+a×tan α)×sin θ

In addition, a movement amount ΔY′ of the top plate 11 along the Y direction at the time of the rotation of the coordinate system by α° is calculated by replacing −D with −(D/cos α+a×tan α) as follows:

ΔY′=a×sin θ−(D/cos α+a×tan α)×(cos θ−1)

Using the rotation matrix of the coordinate system which rotates the coordinate system through −α° can calculate the movement amounts ΔX and ΔY of the top plate 11 by:

$$( \begin{matrix}Δ X \\ Δ Y\end{matrix} ) = ( \begin{matrix}cos θ & sin θ \\ - sin θ & cos θ\end{matrix} ) ( \begin{matrix}Δ {X}^{′} \\ Δ {Y}^{′}\end{matrix} ) = ( \begin{matrix}Δ {X}^{′} \times cos θ + Δ {Y}^{′} \times sin θ \\ - Δ {X}^{′} \times sin θ + Δ {Y}^{′} \times cos θ\end{matrix} )$$

The movement amounts ΔX and ΔY of the top plate 11 are specifically represented by:

ΔX=(D×tan α+a/cos α)×(cos θ−1)+D×sin θ

ΔY=−D×(cos θ−1)+(D×tan α+a/cos α)×sin θ

It is possible to obtain the above equations by calculating coordinates (X, Y) of P2 by rotating P1 by θ, with the coordinates of P1 being given by (a/cos α+D×tan α, −D), and simply calculating ΔX=X−(a/cos α+D×tan α) and ΔY=Y−D.

The top plate control unit 6 moves the top plate 11 in accordance with the movement amounts of the top plate 11 which are calculated by the top plate movement amount calculation unit 5. Moving the top plate 11 will display the designated target region at the same position on the second X-ray image.

### (Top Plate Moving Function)

The top plate moving function is a function of moving the top plate 11, upon the designation of the position of a target region, so as to display the designated target region at the same position in accordance with the rotation of the holding unit 18.

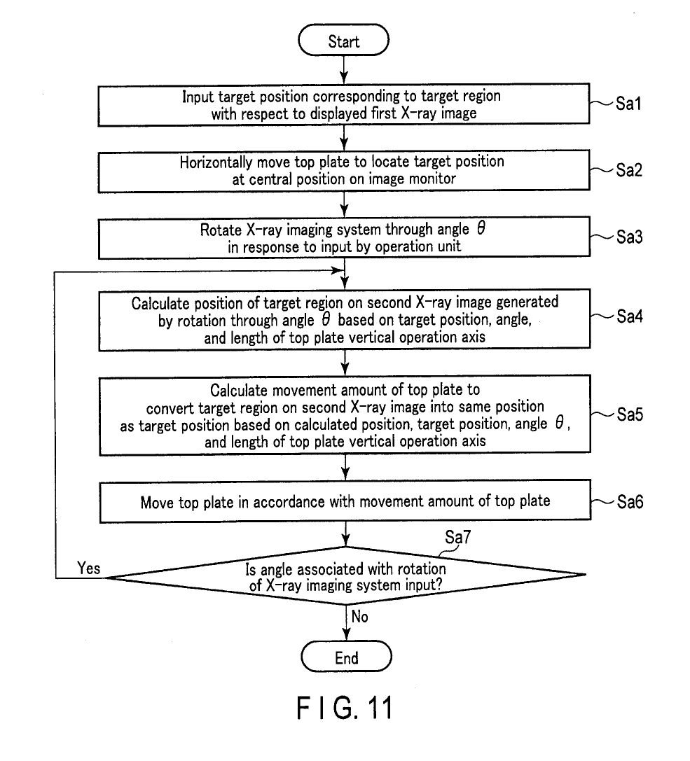

FIG. 11 is a flowchart showing an example of a procedure for the operation associated with the top plate moving function.

A target position corresponding to a target region is input with respect to the displayed first X-ray image (step Sa1). When the target position is to be moved to the central position on the image monitor, the top plate 11 is moved horizontally (step Sa2). Note that when the target position is not to be moved to the central position on the image monitor, the processing in step Sa2 can be omitted.

When the rotation angle θ is input by the operation unit, the X-ray imaging system (holding unit 18) is rotated through the rotation angle θ (step Sa3). The position of the target region on the second X-ray image generated by the rotation through the angle θ is calculated based on the target position, the angle, and the length of the top plate vertical operation axis (step Sa4). The movement amounts of the top plate 11 which cause the target region to be displayed at the same position as the target position on the second X-ray image based on the calculated position, the target position, the angle θ, and the length of the top plate vertical operation axis (step Sa5). The top plate 11 is moved in accordance with the movement amounts of the top plate 11 (step Sa6). If an angle concerning the rotation of the X-ray imaging system (holding unit 18) is input, the processing in steps Sa4 to Sa6 is repeated (step Sa7).

According to the above arrangement, the following effects can be obtained.

This embodiment can provide an X-ray diagnostic apparatus 150 which executes correction of the shift of an imaging position when a member supporting an imaging system is rotated/driven. That is, when a target position corresponding to a target region is input with respect to the first X-ray image, the movement amounts of the top plate 11 are calculated as the holding unit 18 is rotated. This makes it possible to display the target position of the target region at the same position on the image monitor even when the holding unit 18 is rotated.

As described above, there can be provided the X-ray diagnostic apparatus which 150 corrects the shift of an imaging position upon rotating/driving of the member supporting the imaging system by moving the top plate 11, thereby always displaying a target position at the same position without any operation by the operator. This makes it unnecessary for the operator to perform an operation such as moving the top plate 11, and hence improves the diagnostic efficiency with respect to objects.

### (Modification)

A difference from the first and second embodiments is that the movement amounts of the top plate 11 are calculated by considering the value of the shift amount L2 as the difference between the position D of the top plate vertical operation axis, which indicates the distance from the isocenter IC to the top plate 11, and the distance from the top plate 11 to a target position.

The operation unit 8 inputs the distance from the top plate 11 to a target position. Note that the distance from the top plate 11 to the target position may be stored in advance in a memory (not shown) in the main control unit 10.

The target position calculation unit 4 calculates the position of the target region on the second X-ray image based on the distance between the target position and the top plate 11, the rotation angle θ, and the distance from the isocenter and the top plate 11. More specifically, the target position calculation unit 4 calculates the distance between the isocenter and the target position by subtracting the distance between the target position and the top plate 11 from the distance between the isocenter and the top plate 11. The target position calculation unit 4 then calculates the position of the target region on the second X-ray image based on the distance between the isocenter and the target position and the rotation angle θ.

The top plate movement amount calculation unit 5 calculates the movement amounts of the top plate 11 based on the calculated position of the target region, the target position, the rotation angle, and the distance between the target region and the isocenter. More specifically, the top plate movement amount calculation unit 5 calculates the movement amount of the top plate 11 along the longitudinal axis direction by multiplying the distance between the isocenter and the target region by the sine of the rotation angle θ. The top plate movement amount calculation unit 5 also calculates the movement amount of the top plate 11 along the vertical direction by subtracting the product of the distance between the isocenter and the position of the target region and the cosine of the rotation angle θ from the distance between the isocenter and the position of the target region.

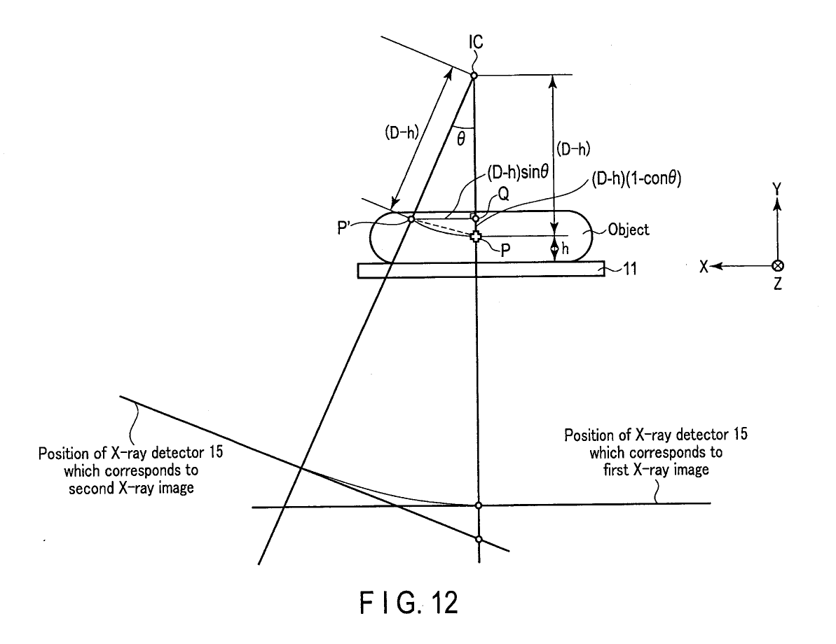

FIG. 12 is a view for explaining the calculation of top plate movement amounts. Referring to FIG. 12, reference symbol “h” denotes the distance between a target region and the top plate 11; “p”, a target position corresponding to the target region; and (D−h), the difference value obtained by subtracting the distance between the target region and the top plate 11 from the distance between the isocenter IC and the top plate 11. As shown in FIG. 12, the movement amount of the top plate 11 along the long axis direction is calculated by multiplying (D−h) by the cosine (sin θ) of the rotation angle θ. As shown in FIG. 12, the movement amount of the top plate 11 along the vertical direction is calculated by subtracting a product (D−h)×cos θ of the distance between the isocenter and the target region and the cosine (cos θ) of the rotation angle θ from the distance (D h) between the isocenter and the position of the target region

{(D−h)−(D−h)×cos θ}.

### (Top Plate Moving Function)

The top plate moving function is a function of moving the top plate 11 to display, upon designation of the position of a target region, the designated target region at the same position in accordance with the rotation of the holding unit 18 and the height from the top plate 11 to a target position.

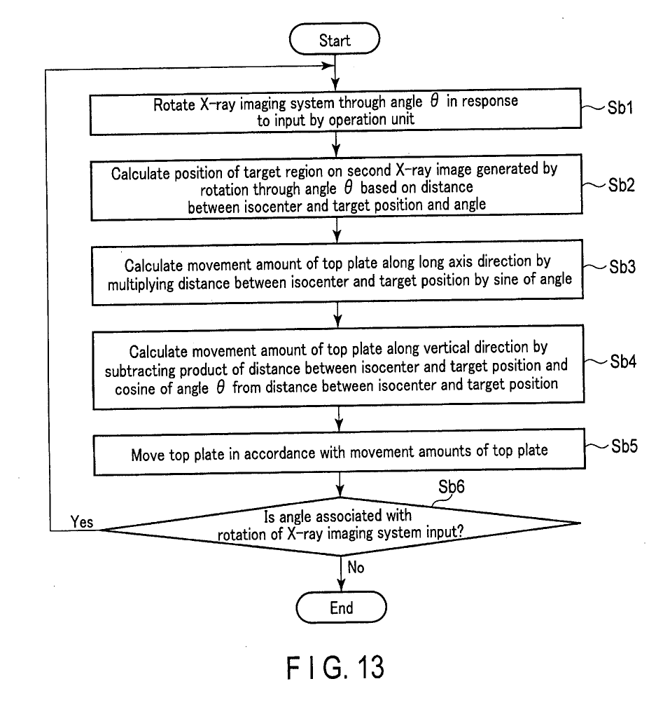

FIG. 13 is a flowchart showing an example of a procedure for the operation associated with the top plate moving function.

When the operation unit inputs the rotation angle θ, the X-ray imaging system (holding unit 18) is rotated through the rotation angle θ (step Sb1). The position of a target region on the second X-ray image generated by the rotation through the rotation angle θ is calculated based on the angle and the distance between the isocenter and the target position (step Sb2). The movement amount of the top plate 11 along the longitudinal direction is calculated by multiplying the distance between the isocenter and the target position by the sine of the angle (step Sb3). The movement amount of the top plate along the vertical direction is calculated by subtracting a product (D−h)×cos θ of the distance between the isocenter and the target position and the cosine of the angle θ from a distance (D−h) between the isocenter and the target position (step Sb4). The top plate 11 is moved in accordance with the movement amounts of the top plate 11 (step Sb5). When an angle concerning the rotation of the X-ray imaging system (holding unit 18) is input, the processing in steps Sb1 to Sb5 is repeated (step Sb6).

According to the above arrangement, the following effects can be obtained.

This embodiment can provide the X-ray diagnostic apparatus 150 which executes correction of the shift of an imaging position when the member supporting the imaging system is rotated/driven. That is, when a target position corresponding to a target region is input with respect to the first X-ray image, the movement amounts of the top plate 11 are calculated in accordance with the distance from the top plate 11 to the target position as the holding unit 18 rotates. This makes it possible to display the target position of the target region at the same position on the image monitor 9 even when the holding unit 18 is rotated.

As described above, there can be provided the X-ray diagnostic apparatus 150 which corrects the shift of an imaging position upon rotating/driving of the member supporting the imaging system by moving the top plate 11, thereby always displaying a target position at the same position without any operation by the operator. In addition, according to this modification, since the movement amounts of the top plate 11 are calculated in accordance with the absolute position of a target region and a rotation angle, the display accuracy of the target region at the target position is improved. This makes it unnecessary for the operator to perform an operation such as moving the top plate 11, and hence improves the diagnostic efficiency with respect to objects. That is, this embodiment can provide an X-ray diagnostic apparatus which executes correction of the shift of an imaging position when a member supporting an imaging system is rotated/driven.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

1. An X-ray diagnostic apparatus comprising:
an X-ray tube and an X-ray detector which face each other;
a holding frame configured to hold the X-ray tube and the X-ray detector such that an imaging system axis passing through a focus of the X-ray tube and a central position of the X-ray detector is configured to rotate around an isocenter as a rotation center around a top plate;
a display configured to display a first X-ray image obtained by the X-ray detector;
operation circuitry configured to input a target position corresponding to a target region with respect to the first X-ray image displayed on the display;
target position calculation circuitry configured to calculate a position corresponding to the target region with respect to a second X-ray image different from the first X-ray image based on an angle between the imaging system axis concerning the second X-ray image and the imaging system axis concerning the first X-ray image, a distance from the isocenter to the top plate, and a distance between the target position and the top plate; and
top plate movement amount calculation circuitry configured to calculate a movement amount of the top plate to display the target region at a same position as the target position on the second X-ray image based on the calculated position, the target position, the angle, and the distance from the isocenter to the top plate.

2. The X-ray diagnostic apparatus according to claim 1, wherein the top plate movement amount calculation circuitry is configured to calculate a movement amount of the target position on the X-ray detector based on the calculated position, the target position, and the angle, and
to calculate the movement amount of the top plate by converting the movement amount of the target position into the movement amount of the top plate using the distance between the top plate and the isocenter.

3. The X-ray diagnostic apparatus according to claim 2, wherein the target position is a central position on the display.

4. The X-ray diagnostic apparatus according to claim 1, wherein the target position calculation circuitry is configured to calculate the position of the target region on the second X-ray image based on the distance between the target position and the top plate, the angle, and the distance between the isocenter and the top plate, and
the top plate movement amount calculation circuitry is configured to calculate the movement amount of the top plate based on the calculated position of the target region, the target position, the angle, and the distance between the target position and the isocenter.

5. The X-ray diagnostic apparatus according to claim 4, wherein the top plate movement amount calculation circuitry is configured to calculate a movement amount of the top plate along a long axis direction by multiplying the distance between the isocenter and the target position by a sine of the angle, and
to calculate a movement amount of the top plate along a vertical direction by subtracting a product of a cosine of the angle and the distance between the isocenter and the target position from the distance between the isocenter and the target position.

6. The X-ray diagnostic apparatus according to claim 1, wherein the operation circuitry is configured to input a position corresponding to the target region on the second X-ray image,
the target position calculation circuitry is configured to calculate a first shift amount indicating a distance between a position corresponding to the target region and the target position on the second X-ray image, and
to calculate a second shift amount indicating a distance between the position corresponding to the target region and the isocenter on the second X-ray image, and
the top plate movement amount calculation circuitry is configured to calculate the movement amount of the top plate based on the first shift amount and the second shift amount.

7. The X-ray diagnostic apparatus according to claim 6, wherein the target position calculation circuitry is configured to calculate the second shift amount by a product of a tangent to the angle and the first shift amount, and
the top plate movement amount calculation circuitry is configured to calculate a movement amount of the top plate along the long axis direction from a product of a sine of the angle and the second shift amount, and
to calculate a movement amount of the top plate along a vertical direction by subtracting a product of a cosine of the angle and the second shift amount from the second shift amount.

8. The X-ray diagnostic apparatus according to claim 1, further comprising top plate control circuitry configured to control movement of the top plate in accordance with the movement amount.
